(19) United States
(12) Patent Application Publication
(10) Pub. No.: US 2005/0277779 A1
(43) Pub. Date: Dec. 15, 2005

(54) Antifungal and antimycobacterial basiliskamides

(75) Inventors: Michael T. Kelly, Surrey (CA); Raymond J. Anderson, Vancouver (CA); Todd A. Barsby, Vancouver (CA)

Correspondence Address: PILLSBURY WINTHROP LLP, Suite 200, 11682 El Camino Real, San Diego, CA, 92130, US

(73) Assignee: University of British Columbia, Vancouver (CA)

(21) Appl. No.: 11/150,607

(22) Filed: Jun. 12, 2002

Related U.S. Application Data
(63) Continuation of application No. 09/585,157, filed on Jun. 1, 2000, now abandoned.
(60) Provisional application No. 60/137,166, filed on Jun. 1, 1999.

(30) Foreign Application Priority Data
Sep. 28, 1998 (WO) PCT/EP98/06146

(51) Int. Cl. C07C 235/46

(52) U.S. Cl. 554/37; 560/104

(57) ABSTRACT

Antibiotic polyketide compounds are provided having the formula 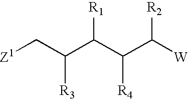 wherein:
R₁ and R₂ are the same or different and are independently H or R;
R is a structural fragment having a saturated or unsaturated linear, branched, or cyclic, skeleton containing one to ten carbon atoms in which the carbon atoms may be optionally substituted with a substituent selected from the group consisting of: —OH; ═O; —OR₅; —O₂CR₅, —SH; —SR₅; —SOCR₅; —NH₂; —NH₅; —NH(R₅)₂; —NHCOR₅; NRCOR₅; —I; —Br; —Cl; —F; —CN; —CO₂H; —CO₂R₅; —CH0; —COR₅; —CONH₂; —CONHR₅; —CON(R₅)₂; —COSH; —COSR₅; —N0₂; —S0₃H; —SOR₅; and —SO₂R₅, wherein R₅ is a linear, branched or cyclic, one to ten carbon saturated or unsaturated alkyl group;
R₃ and R₄ are different and are independently selected from the groups consisting of OH, 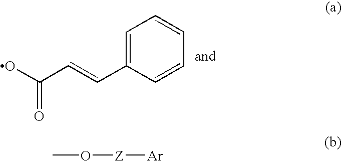 wherein
Z¹ and Z are linear or branched saturated or unsaturated, one to en carbon fragments optionally substituted with Y; Ar is a monocyclic, bicyclic or tricyclic, fully or partially aromatic system containing five or six membered carbocyclic or, oxygen, nitrogen or sulphur containing heterocyclic rings, optionally substituted with R or Y;
Y is selected from the group consisting of: H; ═O, —OH; —OR; —0₂CR; —SH; —SR; —SOCR; —NH₂; —NHR; —NH(R)₂; —NHCOR; NRCOR; —I; —Br; —Cl; —F; —CN— —CO₂H; —CO₂R; —CHO; —COR; —CONH₂; —CONHR: —CON(R)₂; —COSH; —COSR; —N0₂; —SO₃H; —SOR; —SO₂R; and, —O— (epoxide); W is H or R;
   with the provisos that when W is H, R₂ is not H; when R₂ is CH₃, W is not n-propyl; and, one of R₃ and R₄ is (a) or (b) and another of R₃ and R₄ is OH.

### FIELD OF THE INVENTION

This invention relates to polyketide amides having antibiotic activity.

### BACKGROUND OF THE INVENTION

There is an urgent need for new antibiotics to treat pathogens that have developed resistance to antibiotics currently in use. Further, compounds that have antimycobacterial activity are rare. Compounds produced by marine microorganisms are being screened for antibiotic activity.

Japanese patent application 06-27802 published Sep. 12, 1995 under No. 07238018 and entitled “Antimycotic Antibiotic Substance and its Production” discloses an antifungal compound YL-03709B-A obtained by fermentation of Bacillus sp. YL-037093B (FERM P-14126). The Bacillus was isolated from soils near Okinawa, Japan. The compound was reported as having antifungal activity against several organisms but low activity against Candida albicans, Candida parapellosis; Saccharomyces cerevisiae; Saccharomyces sake; and Aspergillus niger on Sabouraud/dextrose Agar medium.

An unidentified Bacillus sp. (MX-PNG-276A) was isolated from the tissues of a tubeworm collected in the tropical waters off Papau, New Guinea. Extracts from laboratory cultures of the latter organism exhibited broad spectrum antibiotic activity against a panel of antibiotic-resistant pathogens. Initial bioassay guided fractionation of crude extracts resulted in the isolation of the loloatins, a family of novel cyclic decapeptides (see PCT/CA9700529). More recently, a class of novel polyketide amides were isolated from MK-PNG-276A cultures, which are termed basiliskamides herein. The basiliskamides have antibiotic activity.

### SUMMARY OF THE INVENTION

This invention provides a compound or a physiologically acceptable salt thereof, wherein the compound has the formula: 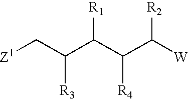
wherein:
R₁ and R₂ are the same or different and are independently H or R;
R is a structural fragment having a saturated or unsaturated linear, branched, or cyclic, skeleton containing one to ten carbon atoms in which the carbon atoms may be optionally substituted with a substituent selected from the group consisting of: —OH; ═O; —OR₅; —O₂CR₅, —SH; —SR₅; —SOCR₅; —NH₂; —NHR₅; —NH(R₅)₂; —NHCOR₅; NRCOR₅; —I; —Br; —Cl; —F; —CN; —CO₂H; —COR₅; —CHO; —COR₅; —CONH₂; —CONHR₅; —CON(R₅)₂; —COSH; —COSR₅; —NO₂; —SO₃H; —SOR₅; and —SO₂R₅, wherein R₅ is a linear, branched or cyclic, one to ten carbon saturated or unsaturated alkyl group;
R₃ and R₄ are different and are independently selected from the groups consisting of OH, 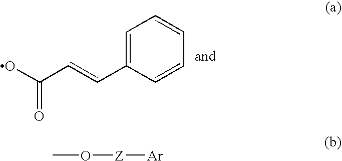
   wherein,
Z¹ and Z are linear or branched, saturated or unsaturated, one to ten carbon fragments optionally substituted with Y;
Ar is a monocyclic, bicyclic or tricyclic, fully or partially aromatic system containing five or six membered carbocyclic or, oxygen, nitrogen or sulphur containing heterocyclic rings, optionally substituted with R or Y;
   Y is selected from the group consisting of: H; ═O, —OH: —OR; —O₂CR; —SH; —SR; —SOCR; —NH₂—, —NHR; —NH(R)₂; —NHCOR; NRCOR; —I; —Br, —Cl; —F; —CN— —CO₂H; —CO₂R; —CHO; —COR; —CONH₂; —CONHR; —CON(R)₂; —COSH; —COSR; —NO₂; —SO₃H; —SOR; —SO₂R; and, —O— (epoxide);
W is H or R;
   with the provisos that when W is H, R₂ is not H; when R₂ is CH₃, W is not n-propyl: and, one of R₃ and R₄ is (a) or (b) and another of R₃ and R₄ is OH.

This invention also provides a compound or a physiologically acceptable salt thereof, wherein the compound has the formula: 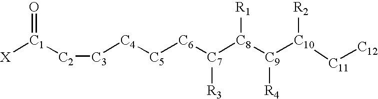
wherein:
a single, double or triple bond exists between one or more of: C-2 and C-3; C-3 and C-4; C-4 and C-5; and C-5 and C-6;
X is NH₂, NHR, NR₂, OH, OR, SH, SR, H, or CF₃;
R is a structural fragment having a saturated or unsaturated linear, branched, or cyclic, skeleton containing one to ten carbon atoms in which the carbon atoms may be optionally substituted with a substituent selected from the group consisting of: —OH; ═O; —OR₅; —O₂CR₅; —SH; —SR₅; —SOCR₅; —NH₂; —NHR₅; —NH(R₅)₂; —NHCOR₅; NRCOR₅; —I; —Br; —Cl; —F; —CN; —CO₂H; —CO₂R₅; —CHO; —COR₅; —CONH₂; —CONHR₅; —CON(R₅)₂; —COSH; —COSR₅; —NO₂; —SO₃H; —SOR₅; and —SO₂R₅, wherein R₅ is a linear, branched or cyclic, one to ten carbon saturated or unsaturated alkyl group:
R₁ and R₂ are the same or different and are independently H or R;
R₃ and R₄ are different and are selected from the group consisting of: OH, 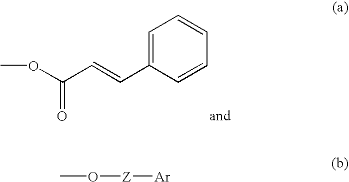
   wherein, Z is a linear or branched, saturated or unsaturated, one to ten carbon fragment optionally substituted with Y;
Ar is a monocyclic, bicyclic or tricyclic, fully or partially aromatic system containing five or six membered carbocyclic or, oxygen, nitrogen or sulphur containing heterocyclic rings, optionally substituted with R or Y;
Y is selected from the group consisting of: H; ═O, —OH; —OR; —O₂CR; —SH; —SR; —SOCR: —NH₂; —NHR; —NH(R)₂; —NHCOR; NRCOR; —I; —Br, —Cl; —F; —CN— —CO₂H; —CO₂R: —CHO; —COR; —CONH₂; —CONHR: —CON(R); —COSH; —COSR; —NO₂; —SO₃H; —SOR: —SO₂R; and —O— (epoxide),
   with the proviso that one of R₃ and R₄ is (a) or (b), and another of R₃ and R₄ is OH.

If a compound of this invention is naturally occurring (such as basiliskamide A or B as described herein) such a compound may be obtained from a natural source or may be sythnesized as described herein. In cases where such a naturally occurring compound is obtained from a natural source, the compound of this invention is characterized as being purified or partially purified. Thus, any compound of this invention that is naturally occurring will be substantially free of cellular contaminants. Cellular contaminants are defined as any component of a living cell (eg. proteins, nucleic acids, cell wall fragments, etc.) or a naturally occurring compound that is not a compound of this invention. The term “substantially free of cellular contaminants” means that a compound or a mixture of compounds of this invention, whether or not present in a pharmaceutical composition, will be present at a ratio of at least 3:1 (w/w) of the total amount of a compound or compounds of this invention to total amount of cellular contaminants present.

This invention also provides pharmaceutical compositions comprising a compound of this invention and a pharmaceutically acceptable carrier selected for the particular indication and mode of treatment in which the compound is to be used.

Compounds of this invention that are capable of forming salts may be in the form of a physiologically acceptable salt. Such a salt is any salt that is acceptable for use in pharmaceutical formulations. For example, where a compound of this invention has a carboxyl or sulphonic acid moiety, the counterion may be Na, K, Mg or Zn. Where the compound comprises a basic moiety such as an amine, the salt may be hydrochloride.

Pharmaceutical compositions of this invention will contain a compound or physiologically acceptable salt thereof in admixture with any carrier, excipient, dilutant, filler, thickener, etc., or in combination with any drug delivery moiety or device selected as suitable for a particular indication and mode of treatment desired. For example, pharmaceutical compositions of this invention may be formulated for injection (intravenous or otherwise), topical application, oral dosage (eg. tablets, capsules, powders), eye drops, aerosol delivery or cosmetic/cleansing formulations such as shampoos or skin cleansers. Selection of pharmaceutically acceptable carriers for compounds of this invention are within the knowledge of those of skill in the art. An example of a formulation for topical use is a creme-based formulation or carrier in which a compound of this invention or a pharmaceutically acceptable thereof is dissolved or emulsified.

This invention also provides a method for treatment of a patient (animal or human) afflicted wilt a fungal or mycobacterial infection comprising the administration to said patient of a therapeutically effective amount or a compound of pharmaceutical composition of this invention. This invention provides the use of a compound or pharmaceutical composition of this invention as an antifungal agent or as an antimycobacterial agent. The dose range of a compound of this invention will be selected in accordance with a particular indication or mode of treatment. Generally, the dose range will be between about 50 and about 500 mg/day for oral and intravenous applications and between about 0.5 and about 5 gram for topical applications.

### DETAILED DESCRIPTION OF EMBODIMENTS OF THE INVENTION

Marine Bacterium MK-PNG-276A: The marine bacterium MK-PNG-276A was isolated during a collecting expedition off of Loloata Island, Papua New Guinea. MIDI analysis of cellular fatty acids indicated that IK-PNG-276A was an unknown species possibly within the genus Bacillus. MK-PNG-276A was deposited Jul. 2, 1996 at the American Type Culture Collection (ATCC) under No. 55797.

Isolation of the Basiliskamides: The marine bacterium MK-PNG-276A was grown in moderate scale culture as confluent lawns for 5 days at 16° C. on trays of solid trypticase soy agar supplemented with NaCl to a final concentration of 1%. The cultures were harvested by gently scraping the cells from the agar surface. Bacterial cells (21.5 g dry weight) were immersed in and subsequently extracted with MeOH (3×250 mL) over a period of six days. Crude MeOH extracts showed broad spectrum antimicrobial activity against a variety of human pathogens, including methicillin resistant Staphylococcus aureus, Eschericia coli, Candida albicans and Mycobacterium tuberculosis.

The combined MeOH extracts were concentrated in vacuo and then partitioned between EtOAc (3×100 mL) and H₂O/MeOH (10:1 200 mL.). The. EtOAc extract was dried over anhydrous Na₂SO₄, filtered and reduced to dryness in vacuo to give 6.5 g of a gum. The gum was fractionated by Sephadex LH-20 chromatography (eluent MeOH) to give 226 mg of a fraction containing strongly UV absorbing compounds. This fraction was subsequently subjected to step gradient reversed-phase chromatography (eluent: 1:1 MeOH/H₂O to 100% MeOH) on a 10 g Waters Sep-Pak. A strongly UV absorbing fraction (82 mg) was further separated into crude basiliskamide A and crude basiliskamide B (28 mg total) by a normal-phase silica gel flash chromatography (4:1 EtOAC/CH₂Cl₃). Final purification was accomplished by reversed-phase HPLC (7:3 MeOH/H₂O), yielding pure basiliskamides A (1, 14 mg) and B (2, 9 mg) as clear solids.

Structure Elucidation of Basiliskamides A (1) and B (2): Basiliskamide A (1) was isolated as a clear solid that gave a [M+H]⁺ ion at m/z 386.23358 in the high resolution fast atom bombardment mass spectrum appropriate for a molecular formula of C₂₃H₃₁NO₄. The ¹³C NMR (Table 1) of basiliskamide A (1) showed only 21 well resolved resonances, indicating that there was an element of symmetry in the molecule. Resonances in the ¹H NMR spectrum of basiliskamide A were all well dispersed, which facilitated identification of the two major substructures.

A broad three proton ¹H NMR resonance at δ 7.40-7.41, that showed HMQC correlations to carbon resonances at δ 128.9 and 130.4, along with a broad two proton ¹H NMR resonance at δ 7.71, that showed HMQC correlations to a carbon resonance at δ 128.4, were all assigned to a monosubstituted phenyl ring. The phenyl ring accounted for the element of symmetry required by the ¹³C NMR data. A one proton doublet at δ 7.65 in the ¹H NMR spectrum showed COSY correlations to the phenyl multiplet at δ 7.71 and to another one proton doublet at δ 6.61. The two doublets were assigned to a vinyl group that was the only substituent on the phenyl ring. HMBC correlations observed between the vinyl doublet resonance at δ 7.65 and the phenyl carbon resonance at δ 128.4 confirmed the attachment of the vinyl group to the phenyl ring. HMBC correlations observed between both of the vinyl proton resonances at δ 7.65 and 6.61 and a carbon resonance at δ 166.0, showed that the phenyl and vinyl fragments were part of a cinnamoyl residue. The vinyl protons had a vicinal scalar coupling of 16 Hz demonstrating the cinnamoyl residue had the E configuration.

Analysis of COSY, HMQC, and HMBC data collected for basiliskamide A (1) routinely identified the linear carbon chain extending from C-2 to C-12, including the positions of the Δ^{2.3} and Δ^{4.5} olefins, the methyl branches at C-8 and C-10, and the presence of —OR substituents at C-7 and C-9. HMBC correlations observed between both the H-2 and H-3 resonances at δ 5.55 and 6.31, respectively, and a carbon resonance at δ 167.5, showed that C-2 was attached to a carbonyl carbon. Only one nitrogen and two hydrogen atoms remained unaccounted for by the cinnamoyl and linear C-1 to C-12 chain fragments, suggesting that the C-1 carbonyl was a primary amide. A pair of broad one proton resonances at δ 6.82 and 7.32, that showed COSY correlations to each other but did not show HMQC correlations to carbon resonances were assigned to the primary amide NH protons. The NH resonance at δ 6.82 showed an HMBC correlation to the C-2 resonance at δ 119.3, confirming the presence of the primary amide at the terminus of the linear C—1 to C-12 carbon chain. A COSY correlation observed between an OH proton resonance at δ 4.57 and the H-7 resonance at δ 3.55 showed that there was an alcohol functionality at H-7 and, therefore, the cinnamoyl fragment had to be attached to the linear carbon chain via an ester linkage at C-9. An HMBC correlation observed between the H-9 methine resonance at δ 4.92 and the cinnamoyl carbonyl resonance at δ 166.0 confirmed the presence of the C-9 ester linkage.

H-2 and H-3 had a vicinal scalar coupling constant of 11 Hz typical of Z olefins, while H-4 and H-5 showed a 15 Hz vicinal coupling typical of E olefins. Difference nOe experiments confirmed the assigned olefinic configurations. Irradiation of the H-3 resonance at δ 6.31 induced an nOe in the H-2 resonance at δ 5.55 in agreement with the Z configuration for the Δ^{2.3} olefin. Similarly, irradiation of the H-5 resonance at δ 5.91 induced a strong nOe in the H-3 resonance at δ 6.31 supporting the E configuration for the Δ^{4.5} olefin.

The relative stereochemistry at C-7 and C-9 was determined by convening basiliskamide A (1) to the acetonide derivative 7. Analysis of the HMQC data for 7, showed that the acetonide methyl carbon resonances had chemical shifts of 19.8 and 30.4 ppm, typical of acetonides formed from syn-1,3-diols Further analysis of the ¹H NMR data for the acetonide 7 showed that the dioxane ring existed in a chair conformation with the C-6 and C-10 carbons equatorial A vicinal coupling constant of 10 Hz was observed between H-9 and H-8 indicating that H-8 was axial and, therefore, the C-14 methyl had to be equatorial, establishing the relative stereochemistries at C-7, C-8, and C-9 as shown in 7. Standard Mosher ester methodology was used to show that C-7 in basiliskamide A (1) had the S configuration. The configuration of C-10 in 1 was not determined. However, basiliskamide A (1) is a homolog of YM147522 (5) and the absolute configuration at C-10 in 5 has been determined by synthesis to be R. Since the other chiral centers in 1 and 5 have identical configurations, and the and the ¹H and ¹³C NMR data for 1 and 5 are nearly identical for the C-7, C-8, C-9 and C-10 centers, it is indicated that 1 also has the R configuration at C-10.

Basiliskamide B (2) was also isolated as a clear solid that gave a [M+H]⁺ ion at m/z 386.23358 in the high resolution fast atom bombardment mass spectrum appropriate for a molecular formula of C₂₃H₃₁NO₄, identical to the formula of basiliskamide A (1). Analysis of the 1D and 2D NMR data obtained for basiliskamide B (2) showed that it was simply an isomer of basiliskamide A, in which the cinnamoyl ester was at C-7 instead of C-9. Basiliskamide B (2) and basiliskamide A (1) were both converted to the same diol 6 by DIBAL reduction, demonstrating that both molecules had identical absolute configurations.

Basiliskamide A (1): isolated as a clear solid; ¹H NMR, see Table 1; ¹³C NMR, see Table 1; IR (film) 98 ₘₐₓ: 3348, 3205, 2966, 2934, 1705, 1697, 1635, 1595, 1450 cm⁻¹; UV (MeOH) λₘₐₓ: 262 nm (ε 41 000); [α]²⁵_{D} (MeOH)=−78; positive-ion HRFABMS [M+H]⁺ m/z 386.23358 (C₂₃H₃₂NO₄, calcd 386.23313).

Basiliskamide B (2): isolated as a clear solid; ¹H NMR, see Table 1; ¹³C NMR, see Table 1; IR (film) 98 ₘₐₓ: 3348, 3205, 2962, 2926, 1702, 1664, 1637, 1595, 1450 cm⁻¹; UV (MeOH) λₘₐₓ: 262 nm (ε 43 000); [α]²⁵_{D} (MeOH)=−12; HREIMS [M]⁺ m/z 385.22531 (C₂₃H₃₁NO₄, calcd 385.22531).

Reduction of Basiliskamides. To basiliskamides B (2ₛ, 4.7 mg) in 1 mL THF under Ar (g), at −78, 4 equivalents of diisobutylaluminum hydride (DIBAL-H) were added. The reaction was stirred overnight then diluted with EtOAc (3 mL) and quenched by the addition of 2 mL NH₄Cl (aq), stirring until the reaction mixture turned cloudy (10 min). The mixture was extracted thrice with EtOAc, and the combined organics were reduced to dryness in vacuo Preparative normal-phase TLC (100% EtOAc) followed by reversed-phas HPLC (70/30 MeOH/H₂O, 280 nm) gave 2 mg of 6. ¹H NMR (500 MHz, DMSO-d₆) δ 7.44 (1H, dd, J=15 Hz, 11 Hz), 7.35 (1H, br s, NH), 6.86 (1H, br s, NH), 6.36 (1H, dd, J=11 Hz), 6.02 (1H, dt, J=15 Hz 7 Hz), 5.57 (1H, d, J=11 Hz), 4.48 (1H, d, J=5.4 Hz, OH), 4.69 (1H, d, J=4.4 Hz, OH), 3.80 (1H, m), 3.23 (1H, m), 2.26 (1H, m), 2.07 (1H, m), 1.61 (1H, m), 1.36 (1H, m), 1.35 (1H, m), 1.18 (1H, m), 0.84 (3H, t, J=7 Hz), 0.72 (3H, d, J=7 Hz), 0.66 (3H, d, J=7 Hz): positive-ion HRFABMS [M+H]⁺ m/z 256.19211 (C₁₄H₂₆NO₃, calcd 256.19127).

Formation of Acetonide (7). To 1.5 mg of 6 in 0.5 mL 2.2-dimethoxypropane, pyridinium p-loluenesulfonate (5 wt % diolbasiliskamide) was added. The reaction mixture was stirred under Ar (g) and heated at 60 C for 1 h. The reaction mixture was filtered through silica (rinsed with EtoAc) and the solvents removed in vacuo. Reversed-phase HPLC (80/20 MeOH/H₂O) yielded 1 mg of 7. ¹H NMR (400 MHz, DMSO-d₆) δ 7.46 (1H, dd, J=15 Hz, 11 Hz, H-4), 7.35 (1H, br s, NH), 6.85 (1H, br s, NH), 6.37 (1H, dd, J=11 Hz, 11 Hz, H-3), 5.94 (1H, dd, J=15 Hz, 7 Hz, H-5), 5.58 (1H, d, J=11 Hz, H-2), 3.57 (1H, m, H-7), 3.48 (1H dd, J=10 Hz, 2 Hz, H-9), 2.44 (1H, m, H-6), 2.19 (1H, m, H-6′), 1.54 (1H, m, H-10), 1.36 (3H, s, Me-17), 1.33 (1H, m, H-8), 1.30 (1H, m, H-11), 1.25 (1H, m, H-11), 1.23 (3H, s, Me-16), 0.83 (3H, t, J=7 HZ, Me-12), 0.75 (3H, d, J=7 Hz, Me-13), 0.71 (3H, d, J=7 Hz, Me-14); ¹³C NMR (DMSO-d₆, 100 MHz) δ 167.8 (C-1), 140.6 (C-3), 138.5 (C-5). 128.9 (C-4), 120.2 (C-2), 97.6 (C-15), 74.9 (C-9). 74.0 (C-7), 36.5 (C-6), 35.1 (C-8), 34.6 (C-10), 30.4 (C-16), 26.7 (C-11), 19.8 (C-17), 12.7 (C-13), 12.1 (C-12), 11.6 (C-14); positive-ion HRFABMS [M+H]⁺ m/z 296.22198, C₁₇H₃₀NO₃, calcd 296.2257.

Reaction of 1 with (R)-MTPA Acid. To a solution of 1 (1.5 mg) in 0.5 mL dry CH₂Cl₂ were added DMAP (1 mg), a drop of triethylamine and (R)-MTPA acid (4 mg) and the solution stirred for 16 h. Removal of solvent in vacuo, followed by preparative reversed-phase TLC (100% MeOH), then reversed-phase HPLC (MeOH/H2O 4:1) gave the (R)-MTPA ester 1a (0.8 mg). ¹H NMR (500 MHz. DMSO-d₆) δ 7.72 (3H, br envelope), 7.43 (9H, br envelope), 7.35 (1H, br s). 6.84 (1H, br s), 6.70 (1H, d, J=16 Hz), 6.20 (1H, dd, J=11 Hz, 11 Hz), 5.58 (2H, m), 5.17 (1H, m), 4.98 (1H, m), 3.43 (3H, s), 2.60 (1H, m), 226 (2H, br m), 1.72 (1H, m), 1.29 (2H, br m), 1.17 (1H, m), 0.95 (3H, d, J=7 Hz), 0.93 (3H, d, J=7 Hz), 0.88 (3H, t, J=7 Hz); positive-ion HRFABMS [M+H]⁺ m/z 602.27148, C₃₃H₃₉NO₆F₃, calcd 602.272950.

Reaction of 1 with (S)-MTPA Acid. To a solution of 1 (1.5 mg) in 0.5 mL dry CH₂Cl₂ were added DMAP (1 mg), a drop of triethy lamine and (S)-MTPA acid (4 mg) and the solution stirred for 16 h. The reaction was quenched and purified as above yielding the (S)-MTPA ester 1b (0.4 mg). 1) NMR (500 MHz, DMSO-d₆) δ 7.72 (3H, br envelope), 7.54 (1H, m), 7.43 (8H, br envelope), 7.38 (1H, br s), 6.91 (1H, br s), 6.72 (1H, d, J=16 Hz), 6.36 (1H, dd, J=11 Hz, 11 Hz), 5.78 (1H, m), 5.64 (1H, d, J=11 Hz), 5.13 (1H, m), 4.94 (1H, m), 3.42 (3H, s), 2.63 (1H, br m), 2.35 (1H, m), 2.17 (1H, m), 1.65 (1H, m), 1.27 (2H, br m), 1.15 (1H, m), 0.89 (3H, d, J=7 Hz), 0.87 (3H, t, J=7 Hz), 0.70 (3H, d, J=7 Hz); positive-ion HRFABMS [M+H]⁺ m/z 602.27352, C₃₃H₃₉NO₆F₃, calcd 602.272950.

TABLE 1
¹³C (100 MHz) and ¹H (500 MHz) NMR Spectal Data
for Basiliskamides A and B in DMSO-d₆

| | Basiliskamide A (1) | | Basiliskamide B (2) | |
|---|---|---|---|---|
| | | δ ¹H | | δ ¹H |
| Atom | δ ¹³C | (intgm, m, J(Hz)) | δ ¹³C | (intgm, m, J(Hz)) |
| 1 | 167.5 | | 167.4 | |
| 2 | 119.3 | 5.55(1H, d, 11) | 119.9 | 5.57(1H, d, 11) |
| 3 | 140.5 | 6.31(1H, dd, 11. 11) | 140.0 | 6.33(1H, dd, 11, 11) |
| 4 | 128.2 | 7.40(1H, m) | 128.8 | 7.51(1H, dd, 15, 11) |
| 5 | 140.5 | 5.91(1H, dt, 15, 7) | 138.0 | 5.87(1H, dt, 15.7) |
| 6 | 34.7 | 2.28(1H, m) | 31.8 | 2.53(1H, m) |
| 6′ | | 1.99(1H, m) | | 2.36(1H, m) |
| 7 | 69.6 | 3.49(1H, m) | 73.0 | 5.40(1H, dt, 10.5, 3) |
| 8 | 40.7 | 2.03(1H, m) | 39.4 | 1.92(1H, m) |
| 9 | 76.3 | 4.92(1H, dd, 9.5, 2) | 74.0 | 3.26(1H, m) |
| 10 | 35.5 | 1.67(1H, m) | 36.3 | 1.40(1H, m) |
| 11 | 26.4 | 1.25(1H, m) | 26.5 | 1.38(1H, m) |
| 11′ | | 1.11(1H, m) | | 1.21(1H, m) |
| 12 | 10.1 | 0.87(3H, t, 7.5) | 11.8 | 0.85(3H, t, 7) |
| 13 | 11.6 | 0.84(3H, d, 7) | 10.7 | 0.83(3H, d, 7) |
| 14 | 12.8 | 0.90(3H, d, 7) | 12.1 | 0.74(3H, d, 7) |
| 15 | 166.0 | | 165.5 | |
| 16 | 118.0 | 6.61(1H, d, 16) | 118.5 | 6.59(1H, d, 16) |
| 17 | 144.6 | 7.65(1H, d, 16) | 144.1 | 7.60(1H, d, 16) |
| 18 | 134.0 | | 134.0 | |
| 19 | 128.4 | 7.71(2H, m) | 128.2 | 7.70(2H, m) |
| 20 | 128.9 | 7.41(2H, m) | 129.0 | 7.40(2H, m) |
| 21 | 130.4 | 7.40(1H, m) | 130.2 | 7.40(1H, m) |
| NH₂ | | 7.31, 6.83(2H, s) | | 7.34, 6.86(2H, s) |
| OH | | 4.57(1H, d, 5) | | 4.48(1H, m) |

### Structures of Basilikamides and Derivatives

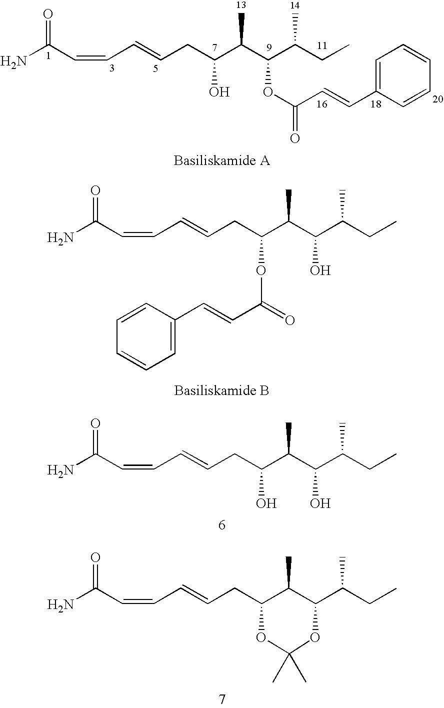

Preparation of Basiliskamides: Compounds of this invention may be prepared from a natural source by fermentation as described above, or by total synthesis, for example by modification of the total synthesis of YM47522 that was described in Ermokenko. M.S. Tetrahedron Letters, 1996, 37, 6711-12 (as exemplified in the scheme below for basiliskamide A).

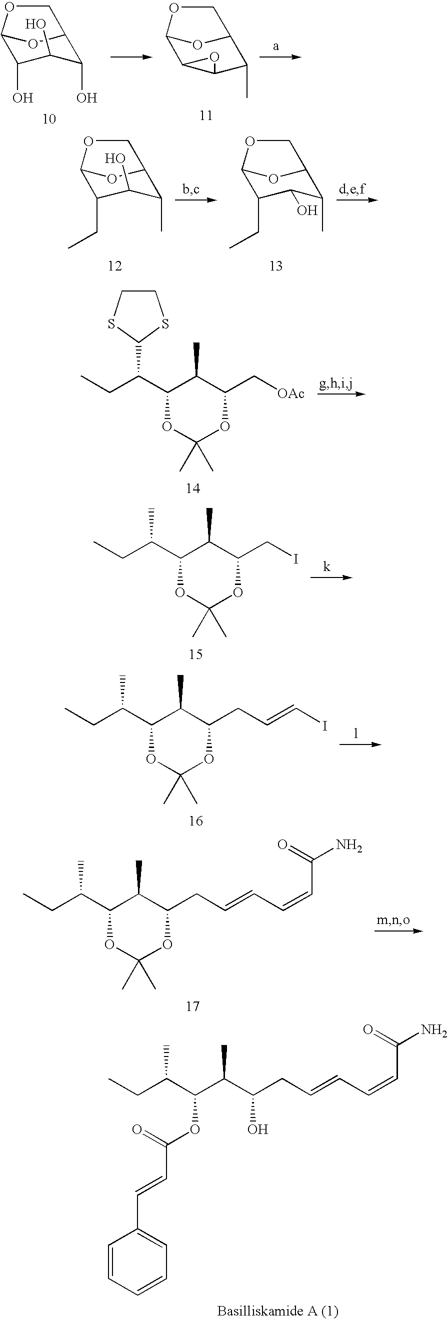

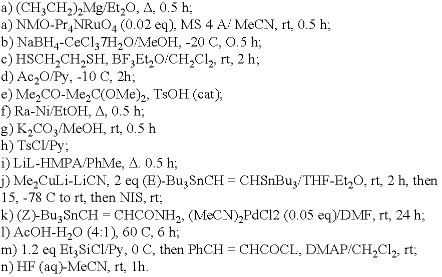

Analogs can be prepared by modification of the total synthesis shown above or by semisynthesis from a product of the total synthesis or a naturally derived product. An example employing basiliskamide A (1) is shown in the scheme below.

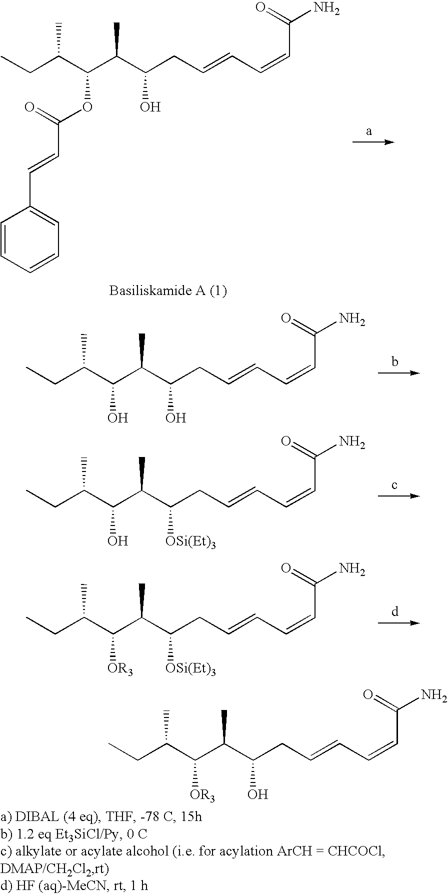

Antifungal Activity of the Basiliskamides: The antifungal activity of basiliskamides A and B was compared to that of the known antifungal agent, amphotericin B. A standardized macrobroth dilution method as used which was developed and published by the National Committee for Clinical Laboratory Standards (Reference Method for Broth Dilution Antifungal Susceptibility Testing of Yeasts, Approved Standard 1996 M27-A, Vol. 15, No. 10). An agar method based on the standardized broth method was also used. The results are presented in Tables 2-5 below.

TABLE 2
Antifungal Activity of Basiliskamide A and B
Tested by Macrobroth Dilution
Minimal Inhibitory Concentration (μg/ml)

| | Candida | Trichophyton | Aspergillus |
|---|---|---|---|
| Basiliskamide | albicans | rubrum | fumigatus |
| A | 0.5 | 1.0 | 4.0 |
| B | ≧32 | 4.0 | ≧32 |

TABLE 3
Antifungal Activity of Basiliskamide A and B
Tested by Agar Dilution
Minimal Inhibitory Concentration (μg/ml)

| | Candida | Trichophyton | Aspergillus |
|---|---|---|---|
| Basiliskamide | albicans | rubrum | fumigatus |
| A | 1.0 | 0.5 | 2.5 |
| B | 3.1 | 5.0 | 5.0 |

TABLE 4
Activity of Basiliskamide A Compared to Amphotericin B
Against 7 Clinical Isolates of Candida albicans
as Determined by Macrobroth Dilution
Minimal Inhibitory Concentration (μg/ml)

| Isolate Number | Basiliskamide A | Amphotericin B |
|---|---|---|
| 8167 | 0.5 | 0.5 |
| 8362 | 0.5 | 0.5 |
| 8363 | 0.5 | 0.5 |
| 8364 | 0.5 | 0.5 |
| 8365 | 0.5 | 0.5 |
| 8366 | 0.5 | 0.5 |
| 8367 | 0.5 | 0.5 |

TABLE 5
Comparative Activity of Basiliskamides A, B and
YL-03709B-A as Determined by Agar Dilution
Minimal Inhibitory Concentration (μg/ml)

| Target | | | Reported Value |
|---|---|---|---|
| Organism | Basiliskamide A | Basiliskamide B | for YL-03709B-A |
| Candida | 1.0 | 3.1 | 25 |
| albicans | | | |
| Aspergillus | 2.5 | 5.0 | ≧50 |
| fumigatus | | | |

These findings demonstrate that the basiliskamides have superior antifungal activities are active against the dermatophyte, Trichophyton rubrum, the yeast. Candida albicans, and the opportunistic fungus, Aspergillus fumigatus. Basiliskamide A activity against clinical isolates of the yeast, Candida albicans when tested by the broth dilution method is comparable to that of amphotericin B, a commonly used antifungal agent. The data presented in Table 5 demonstrates that Basiliskamide A is about 25× more active than YL-03709B-A against the yeast Candida albicans and the filamentous fungus Aspergillus fumigatus, in view of the information for YL-03709B-A reported in Japanese patent application 06-27802. Basiliskamide B also is significantly more active against these organisms than YL-03709B-A.

Antimycobacterial Activity of the Basiliskamides: The Basiliskamides were tested for antimycobacierial activity using a standardized agar dilution method (Underlied, C B and Salfinger, S. 1995. In Manual of Clinical Microbiology, Murray, Baron, Pfaller, Tenover, Yolken (Eds.), ASM Press, page 1395-1404). Activity of basiliskamide A, basiliskamide B, and acylated derivatives of the two compounds were tested for activity against Mycobacterium tuberculosis, the cause of tuberculosis, and Mycobacterium avium-infracellulare, an unimportant cause of mycobacterial infections in immunocompromized patients such as those with AIDS. The results are shown in Table 6.

TABLE 6
Antimycobacterial activity of the Basiliskamides
Minimal Inhibitory Concentration (μg/ml)

| | | M. avium- |
|---|---|---|
| Compound | M. tuberculosis | intracellulare |
| Basiliskamide A | 25 | 100 |
| Basiliskamide B | 50 | >100 |
| Acylated A (2-16) | >100 | >100 |
| Acylated B (2-16) | >100 | ≧50 |

These results indicate that basiliskamide A and B each have activity against M. tuberculosis. Basiliskamide A has activity against M. avium-intracellulare, but basiliskamide B appears to be relatively inactive against this organism. Decreasing the number of carbons in the backbone of the molecule may increase activity. Such is the case with the increased antifungal activity of the basiliskamides (A and B) that have one less carbon in the molecule's backbone than YL-03709B-A.

Cytotoxicity Testing of Basiliskamide A: Serial dilutions of basiliskamide A were prepared in cell culture medium and tested for toxicity for normal human fibroblast cells and for human tumor cell line. The effect of basiliskamide (basil) was compared to that of the known antifungal compound amphotericin B (ampho). The appearance of the cells was assessed after 48 hours exposure to the compounds. The results are shown in Table 7. Basiliskamide produced no cytotoxicity for normal human fibroblast cells at concentrations less than 100 μg/ml compared to amphotericin B (ampho) which was toxic at concentrations as low as 25 μg/ml. Against human tumor cells, basiliskamide showed minor toxicity at concentrations above 3 μg/ml. These findings suggested that basiliskamide is less toxic for normal human cells than the widely used amphotericin B.

TABLE 7
Basiliskamide Cytoxicity Testing
Cytopathic Effect (48 hours)

| | | |
|---|---|---|
| Conc | Human Fibroblast | Human Tumor |

| (μg/ml | Basil | Ampho | Basil | Ampho |
|---|---|---|---|---|
| 100 | 2 | 4 | 4 | 4 |
| 50 | 0 | 2 | 1 | 3 |
| 25 | 0 | 2 | 1 | 1 |
| 12.5 | 0 | 1 | 1 | 0 |
| 6.25 | 0 | 0 | 2 | 0 |
| 3.12 | 0 | 0 | 2 | 0 |
| 1.57 | 0 | 0 | 0 | 0 |
| 0.78 | 0 | 0 | 0 | 0 |
| 0 | 0 | 0 | 0 | 0 |

1 - slight change in morphology vs. control
2 - occasional rounding, vacuolization, or granularity
3 - rounding, vacuolization, detachment of 50% of cells
4 - destruction of monolayer

All publications, patents and patent applications referred to herein are hereby incorporated by reference. While this invention has been described according to particular embodiments and by reference to certain examples, it will be apparent to those of skill in the an that variations and modifications of the invention as described herein fall within the spirit and scope of the attached claims.

1. A compound or a physiologically acceptable salt thereof, wherein the compound has the formula: 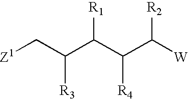
wherein;
R₁ and R₂ are the same or different and are independently H or R;
R is a structural fragment having a saturated or unsaturated linear, branched, or cyclic, skeleton containing one to ten carbon atoms in which the carbon atoms may be optionally substituted with a substituent selected from the group consisting of: —OH; ═O; —OR₅; —O₂CR₅, —SH; —SR₅; —SOCR₅; —NH₂; —NHR₅; —NH(R₅)₂; —NHCOR₅; NRCOR₅; —I; —Br; —Cl; —F; —CN; —CO₂H; —CO₂R₅; —CHO; —COR₅; —CONH₂; —CONHR₅; —CON(R₅)₂; —COSH; —COSR₅; —NO₂; —SO₃H; —SOR₅; and —SOR₂R₅, wherein R₅ is a linear, branched or cyclic, one to ten carbon saturated or unsaturated alkyl group;
R₃ and R₄ are different and are independently selected from the groups consisting of OH, 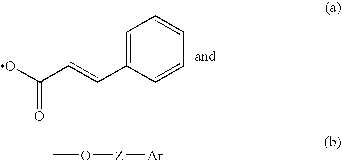
wherein,
Z¹ and Z are linear or branched, saturated or unsaturated, one to ten carbon fragments optionally substituted with Y;
Ar is a monocyclic, bicyclic or tricyclic, fully or partially aromatic system containing five or six membered carbocyclic or, oxygen, nitrogen or sulphur containing heterocyclic rings, optionally substituted with R or Y;
Y is selected from the group consisting of: H; ═O, —OH; —OR; —O₂CR; —SH; —SR; —SOCR; —NH₂; —NHR; —NH(R)₂; —NHCOR; NRCOR; —I; —Br; —Cl; —F; —CN— —CO₂H; —CO₂R; —CHO; —COR; —CONH₂; —CONHR; —CON(R)₂; —COSH; —COSR; —NO₂; —SO₃H; —SOR; —SO₂R; and, —O— (epoxide);
W is H or R;
with the provisos that when W is H, R₂ is not H; when R₂ is CH₃, W is not n-propyl; and, one of R₃ and R₄ is (a) or (b) and another of R₃ and R₄ is OH.

2. The compound or physiologically acceptable salt thereof of claim 1 having the stereoisomeric form:

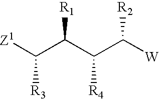

3. The compound or physiologically acceptable salt thereof of claim 1 or 2 wherein Z¹ is a linear or branched, saturated or unsaturated one to eight carbon carbonyl optionally substituted with a substituent selected from the group consisting of: NH₂, NHR, NR₂, OH, OR, SH, SR, H and CF₃, wherein R is as defined.

4. A compound or a physiologically acceptable salt thereof, wherein the compound has the formula: 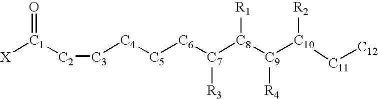
wherein:
a single, double or triple bond exists between one or more of: C-2 and C-3, C-3 and C-4; C-4 and C-5; and, C-5 and C-6;
X is NH₂, NHR, NR₂, OH, OR, SH, SR, H, or CF₃;
R is a structural fragment having a saturated or unsaturated linear, branched, or cyclic skeleton containing one to ten carbon atoms in which the carbon atoms may be optionally substituted with a substituent selected from the group consisting of: —OH; ═O; —OR₅; —O₂CR₅, —SH; —SR₅; —SOCR₅; —NH₂; —NHR₅; —NH(R₅)₂; —NHCOR₅; NRCOR₅; —I; —Br; —Cl; —F; —CN; —CO₂H; —CO₂R₅; —CHO; —COR₅; —CONH₂; —CONHR₅; —CON(R₅)₂; —COSH; —COSR₅; —NO₂; —SO₃H; —SOR₅; and —SO₂R₅, wherein R₅ is a linear, branched or cyclic, one to ten carbon saturated or unsaturated alkyl group;
R₁ and R₂ are the same or different and are independently H or R;
R₃ and R₄ are different and are selected from the group consisting of: OH, 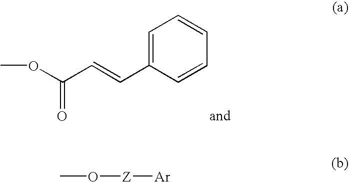
wherein, Z is a linear or branched, saturated or unsaturated, one to ten carbon fragment optionally substituted with Y;
Ar is a monocyclic, bicyclic or tricyclic, fully or partially aromatic system containing five or six membered carbocyclic or, oxygen, nitrogen or sulphur containing heterocyclic rings, optionally substituted with R or Y;
Y is selected from the group consisting of: H; ═O, —OH; —OR; —O₂CR; —SH; —SR; —SOCR; —NH₂; —NHR; —NH(R)₂; —NHCOR; NRCOR; —I; —Br; —Cl; —F; —CN— —CO₂H; —CO₂R; —CHO; —COR; —CONH₂; —CONHR; —CON(R)₂; —COSH; —COSR; —NO₂; —SO₃H; —SOR; SO₂R; and, —O— (epoxide);
with the proviso that one of R₃ and R₄ is (a) or (b), and another of R₃ and R₄ is OH.

5. The compound or physiologically acceptable salt thereof of claim 4 having the structure:

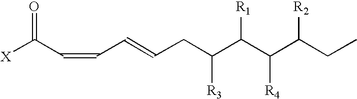

6. The compound or physiologically acceptable salt thereof of claim 4, having the structural and stereoisomeric form:

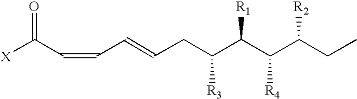

7. The compound or physiological salt thereof of any one of claims 4-6, wherein R₁ and R₂ are independently H or CH₃.

8. The compound or physiological salt thereof of any one of claims 4-7, wherein R₃ is (a).

9. The compound or physiological salt thereof of any one of claims 4-8, wherein X is NH₂.

10. The compound or physiological salt thereof of any one of claims 4-9, wherein R₃ at C₇ is (a) and R₃ at C₉ is OH.

11. The compound or physiological salt thereof of any one of claims 4-9, wherein R₃ at C₇ is OH and R₃ at C₉ is (a).

12. A compound according to claim 4, wherein the compound is Basiliskamide A substantially free of cellular contaminants.

13. A compound according to claim 4, wherein the compound is Basiliskamide B substantially free of cellular contaminants.

14. A pharmaceutical composition comprising a compound or physiological salt thereof of any one of claims 1-13, and a pharmaceutically acceptable carrier.

15. The use of a compound or physiological salt thereof of any one of claims 1-13, as an antifingal agent.

16. The use of a compound or physiological salt thereof of any one of claims 1-3, as an antimycobacterial agent.
